(19) United States
(12) Patent Application Publication
(10) Pub. No.: US 2024/0366385 A1
(43) Pub. Date: Nov. 7, 2024

(54) Modular, Multi-Axis Force Sensor for Measuring Joint Contact Forces

(71) Applicant: Wisconsin Alumni Research Foundation, Madison, WI (US)

(72) Inventors: Joshua Roth, Madison, WI (US); Jason DePhillips, Madison, WI (US)

(21) Appl. No.: 18/650,408

(22) Filed: Apr. 30, 2024

Related U.S. Application Data
(60) Provisional application No. 63/463,967, filed on May 4, 2023.

(51) Int. Cl. A61F 2/30

(52) U.S. Cl. CPC A61F 2/30; A61F 2002/30535

(57) ABSTRACT

A device for measuring normal force, shear forces, and location of the center of pressure in a joint, for example, in lateral and/or medial knee compartments, in the preclinical environment and in the surgical environment to balance the contact forces over a full flexion range. Further, in certain embodiments, the device measures active, passive and shear forces over a full flexion range and is adaptable for both total knee arthroplasty and unicompartmental knee arthroplasty.

### CROSS REFERENCE TO RELATED APPLICATION

This application claims the benefit of U.S. Provisional Application No. 63/463,967, filed May 4, 2023, hereby incorporated by reference.

### STATEMENT REGARDING FEDERALLY SPONSORED RESEARCH OR DEVELOPMENT

### BACKGROUND OF THE INVENTION

The present invention relates to a device for measuring joint loads (e.g., force, stress, strain, torque, or the like) and in particular a device suitable for preclinical, intraoperative, and/or in vivo use to measure loads in a joint such as the knee joint.

Total knee arthroplasty (TKA) is the most common treatment for patients with end-stage osteoarthritis (OA). In TKA, the articulating surfaces of the distal end of the femur and proximal end of the tibia are replaced with prostheses providing, e.g., a sliding polished metal and plastic interface. Unicompartmental (partial) knee arthroplasty (UKA) is a common treatment used for patients with OA in one of the knee compartments, most commonly the medial compartment, where there is damage to only part of the knee. In UKA, there is less post-operative pain and a shorter recovery period than the TKA.

The number of patients undergoing TKA is on the rise with recent predictions being that 3.5 million TKAs will be performed annually by 2030 in the United States alone. Unfortunately, approximately 20% of these patients are not satisfied because of stiffness, instability, residual pain, and/or functional limitations in the treated knee. Similarly, UKA demonstrates high rates of failures, with an approximately 10% to 15% rate of conversion to TKA at an average follow up of five years.

A common cause for this dissatisfaction and an indication for revision is improper load balancing of the contact forces and contact locations of the knee joint. Other reasons for revision surgery include bearing wear, loosening, instability, and malalignment and malpositioning. However, bearing wear information and ligament loading information across the joint can inform the preclinical design and evaluation of new implants or surgical techniques and intraoperative load balancing prior to and during knee arthroplasty.

During a knee arthroplasty procedure, the superficial medial collateral ligament (sMCL) and a lateral collateral ligament (LCL), which are located on either side of the knee joint, are preserved to maintain knee stability. A joint distractor can provide information on ligament tensioning of the sMCL and LCL. For example, as described in US 2019/0200900 for “Apparatus for Intraoperative Ligament Load Measurements,” published on Jul. 4, 2019, assigned to current applicant and hereby incorporated by reference.

Despite the well-recognized importance of measuring loading on the knee joint, accurate measurement of normal and shear forces in these joints remains inadequate.

### SUMMARY OF THE INVENTION

The present invention provides a device for measuring contact forces and center of pressure of a joint such as a knee joint, for example, lateral and/or medial compartment contact forces, in the surgical environment to balance the contact forces over a full flexion range of the knee. Further, the device measures compressive and shear forces over a full flexion range of the knee during either passive (i.e., no muscle loads) or active (e.g., stair climbing) loading and is adaptable for both TKA and UKA.

For example, load balancing of the knee ensures that the disproportionately greater loads that are normally transmitted through the medial compartment (compared to the lateral compartment) do not cause uneven wear and early failure of replacement knee joints. Recent studies have indicated that patient reported outcomes are improved if the knee is quantitatively balanced as compared to unbalanced knees.

In one embodiment, following the tibial cut, intra-compartmental load measurements can inform several surgical corrections, including bone recuts, soft-tissue corrections, and implant alignment adjustments, through a full range of motion, especially if asymmetric loads are encountered.

The present invention provides a device that can be universally embedded into various TKA and UKA implants to independently measure medial-lateral (M-L) force, compression-distraction (C-D) force, anterior-posterior (A-P) force, and center of pressure (CoP) in each of the medial and lateral compartments. The device is adaptable for use with standard and novel TKA and UKA implants and corresponding knee loads that would be expected during active loading (e.g., walking, ascending/descending stairs, etc.) and passive loading (e.g., load applied by a clinician) of the knee joint. With knowledge of these force and center of pressure measurements, the clinician can be given information for preclinical use, i.e., design and evaluation of new implants or surgical techniques, intraoperative load balancing, i.e., balancing the contact forces on the knee compartments, and postoperative monitoring of implant loading and patient function.

Calibration of a load measuring device using multi-axis load cells is made difficult by the mechanical coupling between sensing axes, known as crosstalk. When load is applied in one direction, it is common for multi-axis load cells to output a portion of that load in the other axes. This crosstalk is typically minimized through calibration to achieve accurate multi-axis measurements.

The present invention improves upon the preexisting calibration methods and use of a load measurement device to determine loads on a joint as further described below.

In one embodiment, the present invention provides a device that includes a system for measurement of loading on a knee joint extending along a longitudinal axis, the system comprising a load measuring device insertable into the knee joint and supporting a first force sensor detecting a load input on the knee joint along a first axis and producing a first output voltage indicating at least one of normal and shear force on the knee joint; a second force sensor detecting the load input on the knee joint along a second axis displaced from the first axis and producing a second output voltage indicating the at least one of normal and shear force on the knee joint; a third force sensor detecting the load input on the knee joint along a third axis displaced from the first and second axis and producing a third output voltage indicating the at least one of normal and shear force on the knee joint; and a machine learning system having a processing circuit receiving the first, second, and third output voltages to determine and output an output value functionally related to loading on the joint and based on a predetermined relationship between the first, second, and third output voltages and the load input determined by a machine learning training set.

It is thus a feature of at least one embodiment of the invention to provide a preclinical, intraoperative, and in vivo measure of knee joint loading useful during development and during a variety of knee surgical procedures including but not limited to total knee arthroplasty and unicompartmental knee arthroplasty.

The output value may provide a load measurement of at least one of a normal or compression-distraction force, a shear force in two different directions, and the location or coordinates of the center of pressure on the knee joint. In one embodiment, the output value may provide a load measurement of at least one of a medial-lateral force, compression-distraction force, anterior-posterior force, and center of pressure on the knee joint.

It is thus a feature of at least one embodiment of the invention to provide information that is useful for knee joint balancing and thus resulting in more successful knee arthroplasty outcomes.

An output device may output a loading force based on the predetermined relationship between the output voltages and the loading on the joint at a load location.

It is thus a feature of at least one embodiment of the invention to provide information that may be used preclinically to design and evaluate new implants or surgical techniques or intraoperatively to confirm a preoperative plan for load balancing in knee arthroplasty is achieved.

An output device may output a center of pressure on the knee joint based on the predetermined relationship between the output voltage and the loading on the joint at a load location and further including a display simultaneously indicating the anatomical location of the center of pressure. An image may display the center of pressure superimposed on a knee joint image for physician viewing.

It is thus a feature of at least one embodiment of the invention to provide quick visual reference to allow the physician to deduce load values and center of pressure in and between the medial and lateral knee compartments.

It is thus a feature of at least one embodiment of the invention to provide a clear display for use in the surgical suite that can guide a physician while adjusting load balancing during intraoperative procedures.

The machine leaning training set may comprise a load value data set with associated locations of the load data input and an output voltage data set.

The load measuring device may comprise a disk supporting the first force sensor, second force sensor, and third force sensor displaced apart on the disk. The disk may comprise a ring formed of at least three beams, each supporting a force sensor thereon.

It is thus a feature of at least one embodiment of the invention to provide a compact unit that may be used in a single knee compartment and adaptable with various designs of knee implants.

A fourth force sensor may detect the force on the knee joint along a fourth axis displaced from the first, second and third axes and producing a fourth voltage indicating the at least one of normal and shear force on the knee joint; wherein the machine learning system receives the fourth voltage of the fourth force sensor. The disk may comprise a ring of at least four beams, each supporting a force sensor thereon.

It is thus a feature of at least one embodiment of the invention to provide a measurement of compression-distraction or normal force at opposed corners of the device and shear forces in two different directions.

A fifth force sensor may detect the force on the knee joint along a fifth axis displaced from the first, second, third and fourth axes and producing a fifth voltage indicating the at least one of normal and shear force on the knee joint; wherein the machine learning system receives the fifth voltage of the fifth force sensor. A sixth force sensor may detect the force on the joint along a sixth axis displaced from the first, second, third, fourth, and fifth axes and producing a sixth voltage indicating the at least one of a normal and shear force on the joint wherein the machine learning system receives the sixth voltage of the sixth force sensor. The disk may comprise a ring of at least five beams, each supporting a force sensor thereon. The ring may support a top, bottom, left side and right side beam and first and second cross beams may intersect the top, bottom, left side and rights side beams.

It is thus a feature of at least one embodiment of the invention to measure both normal forces and shear forces in two different directions of the device using, for example, a six beam construction.

The first, second, and third force sensors may detect a compression-distraction force on the knee. The first, second, and third force sensors may detect a center of pressure on the knee. At least one of the first, second, and third force sensors may detect a medial-lateral directional force and anterior-posterior directional force on the knee.

It is thus a feature of at least one embodiment of the invention to measure forces in three directions, torque, and a center of pressure measured along two directions using an arrangement of at least three interconnected beams.

The first, second, and third force sensors may each be at least one strain gauge.

It is thus a feature of at least one embodiment of the invention to allow the discrete strain gauges to be installed on the beams of the device to measure bending of the beams representative of strain.

The disk may comprise a ring of at least three beams wherein each of the beams supports at least two upper strain gauges and at least two lower strain gauges. In one embodiment, each of the beams supports two upper strain gauges and two lower strain gauges. In one embodiment, each of the beams supports four upper strain gauges and four lower strain gauges.

It is thus a feature of at least one embodiment of the invention to provide a structural support for a set of gauges to produce a proportional voltage signal.

The two upper strain gauges and two lower strain gauges are wired through at least one Wheatstone bridge circuit. The at least one Wheatstone bridge circuit may be a full, half or quarter Wheatstone bridge circuit.

It is thus a feature of at least one embodiment of the invention to allow the Wheatstone bridge circuit to be wired into the load cell while minimizing noise output.

A method of measuring joint forces for a joint replacement or arthroplasty comprises the steps of: (a) optionally, resecting at least a portion of one bone of the joint for installation of a load measuring device providing a joint interface; (b) installing the load measuring device into the joint; and (c) making measurement of a load input on the joint interface related to at least one of medial-lateral force, compression-distraction force, anterior-posterior force and center of pressure by: (i) applying a normal force or shear force to the load measuring device to generate an input force on the load measuring device; (ii) detecting a first output voltage on the load measuring device at a first sensor along a first axis; (iii) detecting a output voltage on the load measuring device at a second sensor along a second axis displaced from the first axis; (iv) detecting a third output voltage on the load measuring device at a third sensor along a third axis displaced from the first and second axes; (v) applying a predetermined relationship determined by machine learning between the first, second, and third output voltages at the first, second and third sensors, respectively, and loading on the joint; and (vi) processing the first, second, and third output voltages at the first, second and third sensors, respectively, to output a value functionally related to the load input to the load measuring device.

It is thus a feature of at least one embodiment of the invention to train a machine learning system as part of calibrating the load measuring device to produce a machine learning model which deduces accurate load measurements and center of pressure in vivo. This may be done by 1) making a set of measurements in the device and the sensor is calibrated by training the machine learning model to minimize errors between the predicted and actual values or 2) generate a set of model results and virtually calibrate the sensor by training a machine learning model using the simulation results. Then a set of physical measurements are performed to refine the virtual calibration through transfer learning.

These particular objects and advantages may apply to only some embodiments falling within the claims and thus do not define the scope of the invention.

### BRIEF DESCRIPTION OF THE DRAWINGS

FIG. 1 is a perspective view of force sensing units supported on a knee insert that may be temporarily fitted within a human knee joint constructed according to one embodiment of the present invention to enable the measurement of load forces on the knee in surgical procedures such as total knee arthroplasty;

FIG. 2 is a perspective view of the force sensing units of FIG. 1 supported on a tibia insert with the housing of the units over one compartment shown in phantom;

FIG. 3 is a block diagram of the principal electronic components of the embodiment of FIG. 1 including a trained machine learning system created as a part of calibration that computes receiving load values and load locations of the load values based on output voltages;

FIG. 4 is a perspective view of a force sensing unit of FIG. 1 with the outer housing removed and showing the force transducer supporting a number of loading surfaces surrounded by strain gauges;

FIG. 5 is a simplified representation of normal forces on outer beams of the force sensing unit whereby compression is applied to the upper strain gauges and tension is applied to the lower strain gauges;

FIG. 6 is a simplified representation of shear forces on inner cross beams of the force sensing unit whereby compression is applied to one of the upper strain gauges and tension is applied to the other upper strain gauge and tension is applied to one of the lower strain gauges and compression is applied to the other lower strain gauge; and

FIG. 7 is a simulation of load locations of the load data inputs on an articular surface of a standard tibial insert overlaying the force sensing unit to simulate expected loads in vivo; and

FIG. 8 is an example display provided by the force sensing unit of FIG. 1 showing measured loading forces and center of pressure on the joint shown on an image of the joint.

### DETAILED DESCRIPTION OF THE PREFERRED EMBODIMENT

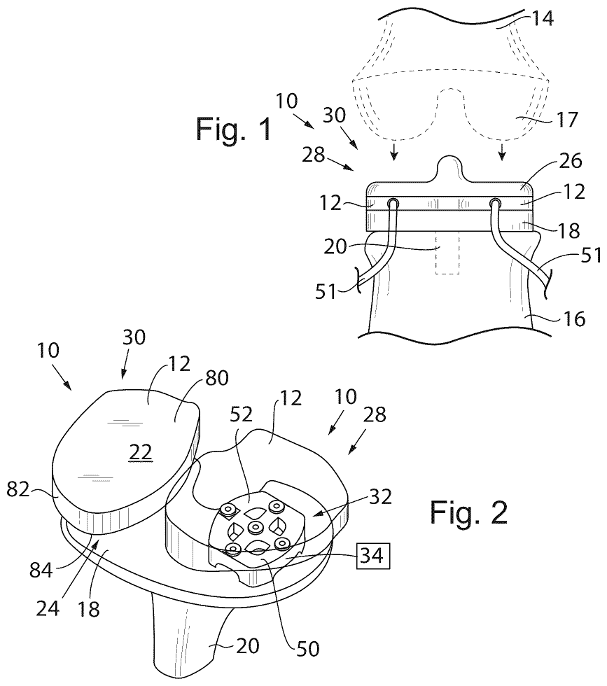

Referring now to FIG. 1 and FIG. 2, a load measuring device 10, constructed according to one embodiment of the present invention, may provide a housing 12 positioned between a femur 14 and a tibia 16 of a patient. In total knee arthroplasty, a distal end of the femur 14 is resected and fit with a femur prosthesis component 17. A similar resection of the tibia 16 may be performed and a corresponding tibia prosthesis baseplate 18 installed over the resected region, for example, fixed by a keel 20 extending axially into the tibia 16.

The housing 12 of the load measuring device 10 has an upper face 22 sized and shaped to receive a plastic liner 26, the plastic liner 26 used to provide a low friction interface between the plastic liner 26 and the femur prosthesis components 17 and that further receives thereon the femur prosthesis component 17 of the patient (or measuring a simulated applied load that would be delivered by the femur prosthesis component 17), and a lower face 24 pressed against or incorporated into the tibia prosthesis baseplate 18.

In a total knee replacement, the tibia prosthesis baseplate 18 has two compartments, a medial compartment 30 (the inside part of the knee) and lateral compartment 28 (the outside part of the knee) which receive the entire femur prosthesis component 17 as shown in FIG. 2. It is understood that FIG. 2 shows a right knee of a patient, but this would be mirrored for a left knee. In a partial knee replacement, the tibia prosthesis baseplate 18 has a single compartment, i.e., the medial compartment 28 or the lateral compartment 30, which receives only a partial femur prosthesis component 17 corresponding with the damaged compartment.

The housing 12 of the measuring unit may be sized to extend over a single compartment, i.e., the medial compartment 28 or the lateral compartment 30, of the tibia prosthesis baseplate 18. In this respect, the tibia prosthesis baseplate 18 for a total knee replacement would support two separate load measuring devices 10 one in each compartment, as seen in FIGS. 1 and 2, and the tibia prosthesis baseplate 18 for a partial knee replacement would support one load measuring device 10 in the single compartment being replaced. The housing 12 may be about 30 mm by 25 mm and less than 30 mm by 25 mm. The discrete, single compartment load measuring device 10 thus allows the device to be adapted for both TKA and UKA usc.

The housing 12 may be a sterile cover, shroud, or sleeve to prevent the transfer of pathogens or damage to the load measuring device 10. The housing 12 may be a disk shaped cover having a top wall 80 and rounded sidewalls 82 corresponding to the top and side of the load measuring device 10. The housing 12 may optionally have a bottom wall 84 corresponding to the bottom of the load measuring device 10 or the bottom wall 84 may be omitted to allow the interior components to be attached directly to or be incorporated into the plastic liner 26 over the tibia prosthesis baseplate 18. The housing 12 may be manufactured out of polyethylene, stainless steel, titanium, and the like. It is understood that the components are chemically sterilizable materials.

Prior to the installation of the load measuring device 10 into the patient's knee, calibration of the load measuring device 10 is performed, for example, using virtual modeling of the load measuring device 10 and applying simulated or virtual loads to the computational model of the load measuring device 10. Or during empirical calibration of the load measuring device 10, a load of the femur 14 and femur prosthesis component 17 can be simulated by applying an actual applied load to the physical support disk 50. Calibration of the load measuring device 10 is described in further detail below.

After calibration of the load measuring device 10 and during use of the load measuring device 10 preclinically, intraoperatively, or in vivo, the load measuring device 10 is installed with the lower face 24 of the housing 12 placed against or incorporated into the tibia prosthesis baseplate 18 and the upper face 22 of the housing 12 installed against the plastic liner 26 to sense actual loads in the knee.

Although the present invention is being described with respect to a tibiofemoral force sensor, it is understood that a similar sensing device may be able to measure the forces of many different joints such as the hip, elbow, shoulder, neck, spine, thumb, wrist, intercarpal joints, patellofemoral joint, and the like.

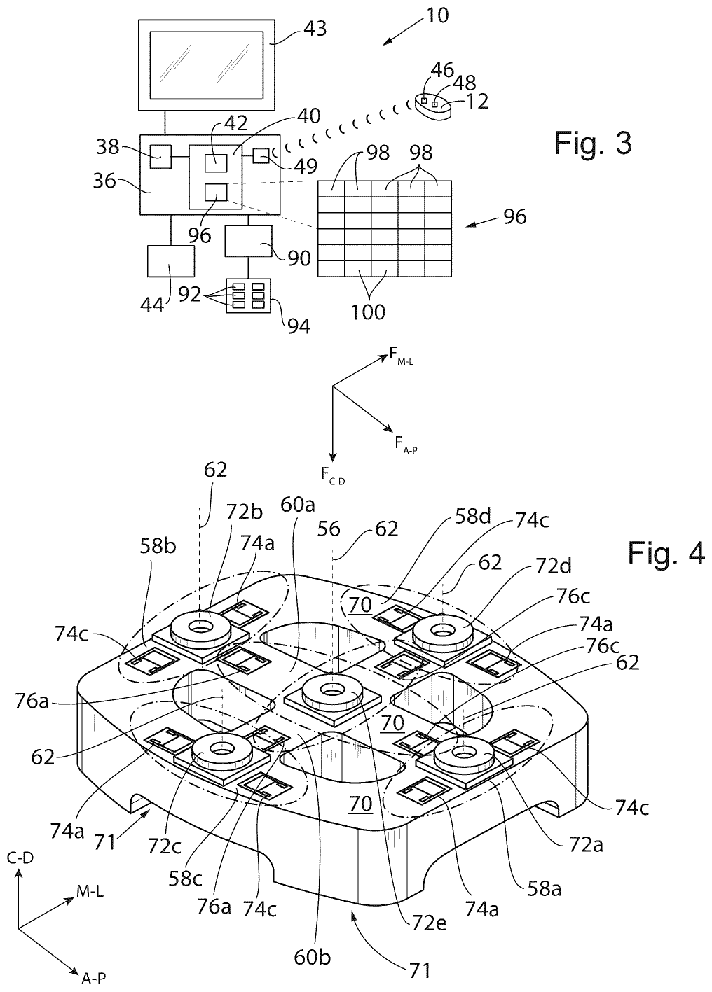

Referring to FIG. 2 and FIG. 3, the housing 12 may support a force transducer 32, for example, multiple load cells, which can convert loads such as tension, compression, pressure, and torque into an electrical signal. The force transducer 32 communicates with a local signal processing circuit 34 which may provide signals to the force transducer 32 from an electronic computer 36 and may sample and digitize the data from the force transducer 32 for transmission to the electronic computer 36. Generally, signals from the force transducer 32 provide a location specific series of force or load data as further described below. The electronic computer 36 may also handle other functions of the load measuring device 10.

The electronic computer 36 may include one or more processors 38 communicating with a memory 40 holding a program 42 as will be described below. In addition, the electronic computer 36 may communicate with the signal processing circuit 34 to send data to the force transducer 32 and to collect data from the force transducer 32 that may also be stored in memory 40 for processing. It is understood that the electronic computer 36 and/or memory 40 may be supported by the housing 12 or be external to the housing 12.

Generally, it will be appreciated that various functions to be described below may be freely allocated between the computer 36 and the local signal processing circuit 34 of the load measuring device 10, and similar allocation can be performed with respect to the location of the machine learning system 94 and storage of data in memory 40. Accordingly, the following description which associates particular functions with a particular location should not be considered a limitation to operation unless context so requires.

The computer processor 38 may communicate with an external graphics display 43 and with the memory 40 holding data values as will be discussed and an internal operating program 42 implementing different display modes to be discussed. The external graphics display communicates with human input controls 44 such as a keyboard, mouse, touchscreen, or the like, allowing a human operator to input data and control the acquisition of data using the present device.

The signal processing circuit 34 may also receive data from the human input controls 44 and may communicate via a wireless link circuit 46 (for example, Bluetooth or Wi-Fi) with a corresponding wireless link circuit 49 associated with the computer 36 as discussed above. The components of the load measuring device 10 may be powered by a contained battery 48 for portable wireless use, as seen in FIG. 3.

Other forms of self-powered energy sources may be employed such as induction and triboelectric or piezoelectric transducers which are able to utilize the applied mechanical stress-tension, compression, and twisting motion at the joint to deliver an electric charge to the load measuring device 10.

Induction powered transducers are described in “Design, calibration and pre-clinical testing of an instrumented tibial tray” found at <https://www.sciencedirect.com/science/article/abs/pii/S0021929007000851?via % 3Dihu b> and “A multiaxial force-sensing implantable tibial prosthesis” found at <https://www.sciencedirect.com/science/article/abs/pii/S0021929005002204?via % 3Dihu b>, which are each hereby incorporated by reference.

The piezoelectric transducers are described in “Energy Harvesting and Sensing with Embedded Piezoelectric Ceramics in Knee Implants” found at <https://www.ncbi.nlm.nih.gov/pmc/articles/PMC6404984/> and “Self-Powered Load Sensing Circuitry for Total Knee Replacement”, found at <https://ieeexplore.ieee.org/document/9529180>, which are each hereby incorporated by reference.

Alternatively, an electrical conductor 51 may allow signals to and from the signal processing circuit 34 to be transmitted to the computer processor 38, as seen in FIG. 1.

Referring to FIG. 2 and FIG. 4, the load measuring device 10 may desirably be calibrated to be further used to evaluate a load or force placed on the knee joint preclinically, intraoperatively or in vivo. In one embodiment, the internal components of the housing 12 may include the force transducer 32 which is a multi-axis load cell defined by a support disk 50 supporting six beams 58 which bend when loads are applied.

Generally, in order to calibrate the load measuring device 10, the force transducer 32 may take repeated measurements of (1) simulated or virtually applied loads applied at different load locations to a computer simulated model of the load measuring device 10 with finite element analysis and/or (2) actual applied loads applied at different load locations to the physical support disk 50. The simulated or virtually applied loads may consider anticipated measurement uncertainty (e.g., electrical noise and gauge malalignment). The input voltage is held constant in order to detect changes in voltage response due to strain differences in the load measuring device 10 and to further determine a relationship between the voltage response and loading and pressure on the device using, e.g., machine learning systems.

Normal and shear forces of the simulated or virtually applied load, or the actual applied load, may pass through the housing 12 to the force transducer 32 on the support disk 50 to convert the resistance on the force transducer 32 due to strain into a voltage response which produces a series of output voltage signals functionally related to location-specific loads on the knee joint. The measurement of voltage signals may be repeated with loads at different load locations (i.e., different loading configurations) over the support disk 50. The measurement of voltage signals may also be used to determine torque load on the support disk 50.

In one embodiment, the support disk 50 has a circular or rectangular outer frame 52 defined by a perimeter of first, second, third and fourth beams 58a, 58b, 58c, 58d formed at front, back, left and right sides, respectively, and surrounding an inner cutout 56 with the inner cutout 56 intersected by first and second cross beams 60a, 60b forming a cross configuration joining the outer frame 52 at 90 degree angles. The first, second, third and fourth beams 58a, 58b, 58c, 58d are joined by the first and second opposed cross beams 60a, 60b such that the first cross beam 60a joins the first beam 58a and second beam 58b and the second cross beam 60b joins the opposed third beam 58c and fourth beam 58d.

The outer frame 52 may be oval thus the beams 58a, 58b, 58c, 58d may be slightly curved or semi-circular. The first and second cross beams 60a, 60b may be generally straight beams. The first beam 58a and second beam 58b may be shorter than the third beam 58c and fourth beam 58d, and the first cross beam 60a may be longer than the second cross beam 60b.

The first beam 58a and second beam 58b are generally equal in length, width, and thickness to provide nearly identical bending moments to each beam in response to a same or similar force. The equal length produces the same bending moments while the equal width and thickness produces the same stresses and strains. The third beam 58c and fourth beam 58d are generally equal in length, width, and thickness to provide nearly identical bending moments to each beam in response to a same or similar force. Again, the equal length produces the same bending moments while the equal width and thickness produces the same stresses and strains. The first and second cross beams 60a, 60b are generally equal in length, width, and thickness to provide nearly identical bending moments to each beam in response to a same or similar force. An upper surface 70 and a lower surface 71 of the outer frame 52 and the first and second cross beams 60a, 60b, respectively, are generally flush surfaces to simultaneously receive the same contact forces.

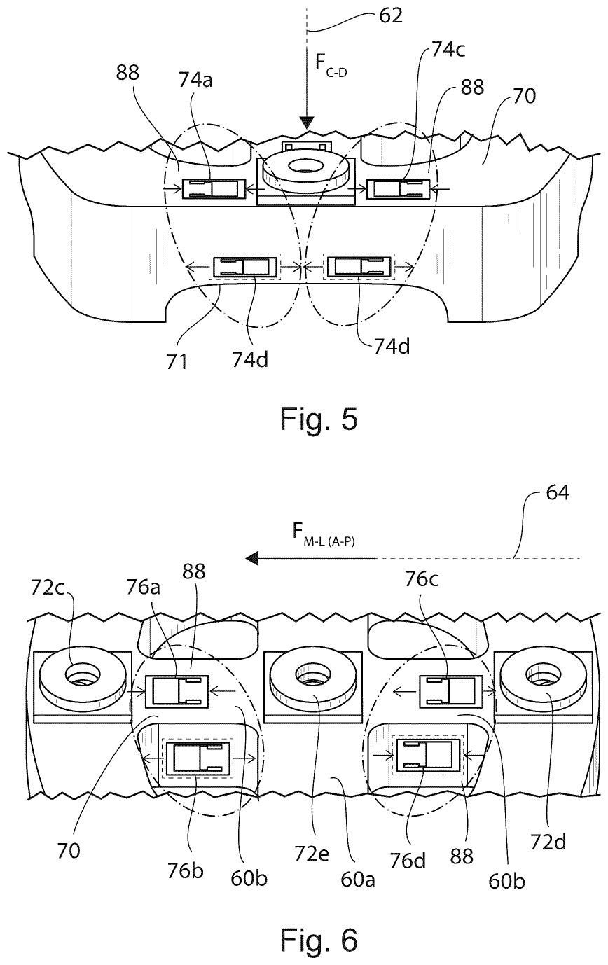

Referring also to FIGS. 5 and 6, a spot loading surface 72 is supported on the upper surface 70 of each of the first, second, third and fourth beams 58a, 58b, 58c, 58d, respectively, proximate the center of a length of each beam. There are four spot loading surfaces 72a, 72b, 72c, 72d which are each defined by a circular stage elevated from the upper surfaces 70 by a circular or rectangular base with the spot loading surfaces 72a, 72b, 72c, 72d desirably receiving a discrete force measurement applied normal (F_{C-D}) to the spot loading surfaces 72a, 72b, 72c, 72d along parallel vertical force axes 62 but also shear forces (F_{M-L} and F_{A-P}) perpendicular to the surface normal along horizontal force axes 64 and perpendicular to the horizontal force axis 64.

An intersection of the first and second cross beams 60a, 60b at the center of the support disk 50 supports a fifth spot loading surface 72e. The fifth spot loading surface 72e is similarly defined by a circular or rectangular base with the spot loading surfaces 72e desirably receiving a discrete force measurement applied normal (F_{C-D}) to the fifth spit loading surface 72e along parallel vertical force axis 62 but also shear forces (F_{M-L} and F_{A-P}) perpendicular to the surface normal along a horizontal force axis 64 and perpendicular to the horizontal force axis 64.

In alternative embodiments, the support disk 50 may include less than six beams 58 such as three, four, or five beams which may be arranged in a triangular or rectangular configuration with one or more cross beams. In alternative embodiments, the support disk 50 may include more than six beams 58 such as seven, eight, or nine beams which may be arranged like spokes of a wheel. Each of these beams may similarly support spot loading surfaces as described above for receiving a discrete force measurement. Each of the beams may be associated with strain gauges as further described below.

A plurality of strain gauges 74 are independently mounted on the support disk 50 and associated with each beam 58a, 58b, 58c, 58d and cross beam 60a, 60b to independently measure the amount of normal force on the beams 58a, 58b, 58c, 58d and shear forces on the beams 58a, 58b, 58c, 58d and cross beams 60a, 60b when a force is applied to the spot loading surfaces 72a, 72b, 72c, 72d, 72e. By mounting strain gauges 74 on the top and bottom of each beam, the resistance on each strain gauge 74 can be translated to an applied load on each beam. Although two upper strain gauges 74a, 74c and two lower strain gauges 74b, 74d are seen and described below, defining a “sensor” of each beam (shown circled in FIG. 4), it is understood that any number of upper and lower strain gauges is contemplated such as four upper strain gauges and four lower strain gauges.

Referring specifically to FIG. 5, in one embodiment, each of the beams 58a, 58b, 58c, 58d and cross beams 60a, 60b are associated with a beam “sensor” having two upper strain gauges 74a, 74c mounted to the upper surface 70 of the beams 58a, 58b, 58c, 58d and flanking the spot loading surfaces 72a, 72b, 72c, 72d and two lower strain gauges 74b, 74d mounted to the lower surface 71 of the beams 58a, 58b, 58c, 58d and flanking the spot loading surfaces 72a, 72b, 72c, 72d. The two upper strain gauges 74a, 74c will detect compression on the beams 58a, 58b, 58c, 58d, e.g., when a compressive normal force (F_{C-D}) is applied to the associated spot loading surface 72a, 72b, 72c, 72d, and the two lower strain gauges 74b, 74d will detect a tension in the beams 58a, 58b, 58c, 58d, e.g., when a compressive normal force (F_{C-D}) is applied to the associate spot loading surface 72a, 72b, 72c, 72d. In this respect, the four spot loading surfaces 72a, 72b, 72c, 72d will receive the force on the first, second, third and fourth beams 58a, 58b, 58c, 58d of the outer frame 52 along the vertical axes 62 when a normal force is applied. In a similar manner, the fifth spot loading surface 72e will receive the force on the two intersecting cross beams 60a, 60b along the vertical axis 62, e.g., when a normal force (F_{C-D}) is applied.

Referring specifically to FIG. 6, the fifth spot loading surface 72e receives the force of the two intersecting cross beams 60a, 60b. The first cross beam 60a will support a beam “sensor” (shown circled in FIG. 4) with two upper strain gauges 76a, 76c mounted to the upper surface 70 of the first cross beam 60a and flanking the fifth spot loading surface 72e and two lower strain gauges 76b, 76d mounted to the lower surface 71 of the first cross beam 60a and fifth spot loading surface 72c. As seen, the second cross beam 60b will support a beam “sensor” (shown circled in FIG. 4) with two upper strain gauges 76a, 76c mounted to the upper surface 70 of the second cross beam 60b and flanking the fifth spot loading surface 72e and two lower strain gauges 76b, 76d mounted to the lower surface 71 of the second cross beam 60b and fifth spot loading surface 72c.

In one embodiment, as shown in FIG. 6, the two upper strain gauges 76a, 76c will detect compression and tension, respectively, on the first and second beams 60a, 60b when a shear force (F_{M-L} or F_{A-P}) is applied to the fifth spot loading surface 72e and the two lower strain gauges 76b, 76d will detect an opposite tension and compression, respectively, of the first and second beams 60a, 60b when a shear force (F_{M-L} or F_{A-P}) is applied to the fifth spot loading surface 72e. In a similar manner, the spot loading surfaces 72c, 72d will receive the forces on the beams 58c, 58d along the horizontal force axis 64 and the spot loading surfaces 72a, 72b, will receive the forces on the beams 58a, 58b along the axis perpendicular to the horizontal force axis 64. It is understood that if the shear force (F_{M-L} or F_{A-P}) switches direction from the direction shown in FIG. 6 then the forces on the beams described above are mirrored.

Generally, the upper and lower strain gauges of the beams 58c, 58d, 60b will detect compression and tension when shear force (F_{M-L}) is applied in the medial-lateral (M-L) direction and the upper and lower strain gauges of the beams 58a, 58b, 60a will detect compression and tension when shear force (F_{A-P}) is applied in the anterior-posterior (A-P) direction.

The upper strain gauges 74a, 74c and the lower strain gauges 74b, 74d of each beam are wired through a Wheatstone bridge circuit 88. In total, there are twenty four strain gauges on the support disk 50 wired through Wheatstone bridge circuits 88 that will produce output voltage for the simulated loads and their load locations (i.e., different loading configurations).

In certain embodiments the strain gauges 74, 76 are commercially available and may be “L2A-13-015LW-120” type manufactured by Micro-Measurements of Wendell, NC. The strain gauges 74, 76 may have a strain range of +3% and overall width and length of approximately 1.37 mm and 1.9 mm.

It is generally understood that the measurement may occur in three directions that are not necessarily defined by anatomical relationships as described above and may be generally defined as a compression-distraction or normal force, shear forces in two directions (e.g., perpendicular directions), and a center of pressure measurement with respect to the two directions (e.g., perpendicular directions).

The Wheatstone bridge circuit 88 is represented by the following general Wheatstone bridge equation. Wheatstone bridge equations take the strain values (¿) and an input voltage (Vᵢₙ) and convert them into an output, in this case an electrical signal, and more specifically, an output voltage (Vₒᵤₜ). The changes in the strain values (¿) associated with loading with a set input voltage (Vᵢₙ) can be measured by changes in the output voltage (Vₒᵤₜ). In one embodiment, the output voltage may be derived according to the following Wheatstone bridge equation modeling the knee joint as bending beams as follows:

$${V}_{out} = \frac{1}{4} {V}_{in} k ( {ε}_{1} - {ε}_{2} + {ε}_{3} - {ε}_{4} ) \qquad ( 1 )$$

$$k = gauge factor$$

$$tensi on ( + ) , compression ( - )$$

It is understood that the Wheatstone bridge circuit 88 can vary, for example, full, half, and quarter Wheatstone bridges whereby varying number of strain measurements are measured. Quarter bridges measure one strain measurement (¿) in the Wheatstone bridge equation. Quarter bridges offer the greatest number of output voltages (Vₒᵤₜ) for a set amount of gauges but a lower sensitivity. Full bridges measure four strain measurements (ε) in the Wheatstone bridge equation. Full bridges are the most sensitive but offer the least number of output voltages (Vₒᵤₜ) for a set amount of gauges. Half bridges measure two strain measurements (ε) in the Wheatstone bridge equation and are further described below with respect to a preferred embodiment. Half bridges may provide a good tradeoff between sensitivity and output voltage (Vₒᵤₜ) while being able to be wired on the support disk 50.

Referring specifically to FIG. 5, in one embodiment, the upper strain gauge 74a and lower strain gauge 74b of each of the first, second, third and fourth beams 58a, 58b, 58c, 58d, respectively, are wired through a first Wheatstone bridge circuit 88 and the upper strain gauge 74c and lower strain gauge 74d of each of the first, second, third and fourth beams 58a, 58b, 58c, 58d, respectively, are wired through a second Wheatstone bridge circuit 88 for each spot loading surface 72a, 72b, 72c, 72d.

As the beams 58a, 58b, 58c, 58d are strained due to the applied compression-distraction force (F_{c}-D), the top strain gauges 74a, 74c are compressed, while the bottom strain gauges 74b, 74d are stretched. The Wheatstone bridge circuit 88 may be represented by the following half Wheatstone bridge equations used to determine an output voltage (Vₒᵤₜ) when there is a compression-distraction force (F_{C-D}) on the respective beams 58a, 58b, 58c, 58d. In a similar manner, the following half Wheatstone bridge equations may be used to determine an output voltage (Vₒᵤₜ) when there is a compression-distraction force (F_{C-D}) on the first and second beams 60a, 60b.

With F_{C-D}:

$${V}_{out} = \frac{1}{4} {V}_{in} k ( {ε}_{1} - ( - {ε}_{2} ) ) \qquad ( 2 )$$

$${V}_{out} = \frac{1}{4} {V}_{in} k ( {ε}_{1} + {ε}_{2} ) \approx \frac{1}{2} {V}_{in} k ε \qquad ( 3 )$$

$${V}_{out} = \frac{1}{4} {V}_{in} k ( {ε}_{3} - ( - {ε}_{4} ) ) \qquad ( 4 )$$

$${V}_{out} = \frac{1}{4} {V}_{in} k ( {ε}_{3} + {ε}_{4} ) \approx \frac{1}{2} {V}_{in} k ε \qquad ( 5 )$$

Referring specifically to FIG. 6, in one embodiment, the upper strain gauge 76a and lower strain gauge 76b of the first and second beams 60a, 60b, respectively, are wired through a first Wheatstone bridge circuit 88 and the upper strain gauge 74c and lower strain gauge 74d of the first and second beams 60a, 60b, respectively, are wired through a second Wheatstone bridge circuit 88 for each beam intersecting the fifth spot loading surface 72e.

As the beam is strained due to the applied medial-lateral force (F_{M-L}) and/or anterior-posterior force (F_{A-P}), the top strain gauge 76a is compressed and the top strain gauge 76c is stretched, while the bottom strain gauge 76b is stretched and the bottom strain gauge 76d is compressed. Therefore, opposite tension and compression occurs across the fifth spot loading surface 72e causing shear stress. The Wheatstone bridge circuit 88 may be represented by the following half Wheatstone bridge equations used to determine an output voltage (Vₒᵤₜ) when there is a medial-lateral force (F_{M-L}) on the second cross beams 60b and anterior-posterior force (F_{A-P}) on the first cross beams 60a. In a similar manner, the following half Wheatstone bridge equations may be used to determine an output voltage (Vₒᵤₜ) when there is a medial-lateral force (Fra) on the beams 58a, 58b and anterior-posterior force (F_{A-P}) on the beams, 58c, 58d.

With F_{M-L}, A-P.

$${V}_{out} = \frac{1}{4} {V}_{in} k ( {ε}_{1} - {ε}_{2} ) \qquad ( 6 )$$

$${V}_{out} = \frac{1}{4} {V}_{in} k ( - {ε}_{1} - {ε}_{2} ) \approx - \frac{1}{2} {V}_{in} k ε \qquad ( 7 )$$

$${V}_{out} = \frac{1}{4} {V}_{in} k ( {ε}_{3} - {ε}_{4} ) \qquad ( 8 )$$

$${V}_{out} = \frac{1}{4} {V}_{in} k ( {ε}_{3} - ( - {ε}_{4} ) ) \approx \frac{1}{2} {V}_{in} k ε \qquad ( 9 )$$

The constant input voltage (Vᵢₙ) and measured strain values at each strain gauge 74, 76 are used to calculate the output voltage (Vₒᵤₜ) using quarter, half, and/or full Wheatstone bridge equations.

Calibration of the load measuring device 10 can determine a functional relationship between the output voltage (Vₒᵤₜ) and the load values and their load locations to estimate the loads (i.e., medial-lateral (M-L) force, compression-distraction (C-D) force, anterior-posterior (A-P) force), torque, and center of pressure (CoP) on the load measuring device 10, for example, preclinically or when measured intraoperatively or in vivo.

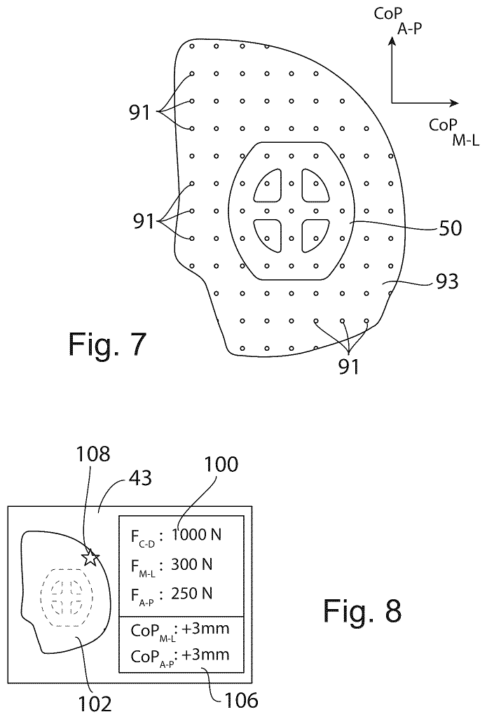

Referring now to FIG. 7, initial calibration of the load measuring device 10 may involve a computer simulation of the support disk 50 with a simulated or virtual excitation applied to the computer model of the support disk 50, or empirically applying an actual applied load to the physical support disk 50. The load magnitudes may represent an expected in vivo force range of 0 to 4000 N for C-D force (F_{C-D}) (i.e., active forces 0 to 4000 N and passive forces 0 to 500 N) and an expected in vivo force range of 0 to 360 N medial-lateral force (F_{M-L}) and anterior-posterior force (F_{A-P}) (i.e., shear forces). The load values may represent both negative and positive force values.

The load locations 91 may be varied over the expansive footprint 93 representative of the tibia prosthesis baseplate 18 which supports the support disk 50 thereon and is larger than the footprint of the support disk 50 itself to simulate active and passive load locations on the tibia prosthesis baseplate 18 of the knee implant over the support disk 50.

Referring also to FIG. 3, the constant input voltages (Vᵢₙ) and measured strains at different loading configurations (i.e., different load values at different load locations 91 described above) are input into the Wheatstone bridge equations to determine an output voltage (Vₒᵤₜ).

A relationship between the output voltage (Vₒᵤₜ), determined from the Wheatstone bridge equations, and the corresponding loads (i.e., medial-lateral (M-L) force, compression-distraction (C-D) force, anterior-posterior (A-P) force), torque, and center of pressure (CoP) with respect to two directions (i.e., medial-lateral (M-L) and anterior-posterior (A-P)) on the support disk 50 can be determined using a transfer learning approach or model, for example, a single sensitivity matrix like a matrix algebra model using the least square method, multiple linear regression model with interactions, or machine learning systems like a neural network model or deep learning model.

An exemplary method of a simulated or virtual calibration step is described below with respect to Example 1.

### Example 1—Virtual Calibration of the Force Transducer

In one embodiment, a computer-aided-design (CAD) and finite element (FE) model of the support disk 50 described above is produced.

An excitation voltage (Vᵢₙ) of 5 V and amplifier are set to achieve an output of 10 V (Vₒᵤₜ) at maximum load. The load locations of the applied forces or loads may include 79 locations subjected to 500 loading configurations to give a complete dataset of 40,000 load values and associated locations of the load data inputs.

From the 40,000 data points, the respective normal strains of the twenty four strain gauges 74 are extracted and input into Wheatstone bridge equations described above. The amount of noise to the output signal is estimated by adding virtual electrical noise using a normal distribution (mean: 0; SD: 1.5 mV) and the signal amplified to achieve an output of 10 V (Vₒᵤₜ) at maximum loading. The amount of noise added is based on the expected electrical noise of signal conditioning electronics (NI 9237, National Instruments).

For each 40,000 data point inputs, the full, half, and quarter Wheatstone bridge configurations produce six, twelve and twenty-four output voltages (Vₒᵤₜ), respectively. The performance of calibration algorithms is directly proportional to the number of output voltages (Vₒᵤₜ).

The output voltages (Vₒᵤₜ), load values, and associated location of load data inputs, will produce a complete training set 90. The complete training set 90 is used for training (and optionally, testing) the different linear algebra, matrix algebra, and machine learning models described below. The models may be built using the complete data set of 40,000 simulations or a partial data set. In one embodiment, the models may be built in MATLAB. It is understood that other software or custom code may be used.

Exemplary models described below may be processed to output predicted loads (i.e., medial-lateral (M-L) force, compression-distraction (C-D) force, anterior-posterior (A-P) force) on the beams at a center of pressure (CoP) of the support disk 50 with respect to two directions (i.e., medial-lateral (M-L) and anterior-posterior (A-P)) to the reviewing clinician.

### Example 2: Matrix Algebra Model

The matrix algebra model is the most traditional means to calibrate sensors by relating independent variables to dependent variables through a sensitivity coefficient matrix. This can be represented by the equation below.

$$A * X = B \qquad ( 10 )$$

For example: A is a 5×W matrix of inverse sensitivity coefficients. The 5 rows represent the number of sensor measurements: force in all three directions and center of pressure in two directions. If torque or another measurement was added, then the matrix A would have different dimensions.

W varies with respect to the Wheatstone bridge configuration of the strain gauges (full bridge: W=6; half bridge: W=12; quarter bridge: W=24). If there were a different number of beams, there would be a different number of output voltages W.

X is a matrix of the dependent variables, also known as the output voltages of the sensor (W×40,000). The 40,000 columns represent the different loading configurations. B is a matrix of the independent variables, also known as the force in all three directions and center of pressure in two directions.

Given B and X, one can solve for the inverse sensitivity coefficient matrix, A, with units of load/voltage or location/voltage (for the center of pressure) using code written and solved in MATLAB.

$$A = B * {X}^{- 1} \qquad ( 11 )$$

### Example 3: Linear Regression with Interactions

The linear regression with interactions model has a similar concept but incorporates additional sensitivity coefficients that interact between two or more of the output voltages (Vₒᵤₜ).

Linear regression analysis is used to predict the value of a variable based on the value of another variable. In multiple linear regression, the goal is to attempt to model the linear relationship between certain input variables and an output variable using sensitivity coefficients. When interactions are involved, there are additional sensitivity coefficients that interact between two or more of the independent variables.

In one embodiment, MATLAB's regression learner app is used to estimate the coefficients and interactions that best relate the dependent variables to the independent variables. It is understood that other software or custom code may be used.

### Example 4: Machine Learning/Neural Network

Neural networks learn to recognize patterns in the data to minimize prediction errors. In one embodiment, the machine learning system 94 may employ the framework of a series of algorithms that make up layers. The trained models consist of MATLAB's default value of 100 layers. The first fully connected layer of the neural network has a connection from the network input (output voltage data), and each subsequent layer has a connection from the previous layer. Each fully connected layer multiplies the input by a weight matrix and then adds a bias vector. An activation function follows each fully connected layer, excluding the last. The final fully connected layer produces the network's output (sensor measurements), namely predicted response values.

In one embodiment, MATLAB's regression learner app is used to build the neural network model. Default settings were found to be optimal for this application. It is understood that other software or custom code may be used.

Using this neural network framework, load values, the associated load locations of the load values, and corresponding output voltages (Vₒᵤₜ) are provided to the machine learning system 94 to provide a relationship between the output voltage (Vₒᵤₜ) and load values and center of pressure (CoP) so that unknown load values and center of pressure (CoP) may be determined from sensor measurements based on weights 92 derived during training.

The machine learning system 94 may be trained with the teaching set comprised of load values, the associated load locations of the load values, and corresponding output voltage (Vₒᵤₜ). The sensor measurements are output voltage (Vₒᵤₜ) which are correlated to load values and center of pressure (CoP).

The various components described above may be implemented on appropriate hardware, for example, as used for machine calculations and machine learning as is understood in the art.

Referring to FIG. 3 and FIG. 8, in operation, the trained algorithms may be used to predict unknown applied active or passive loads, for example, that may be applied preclinically, intraoperatively or in vivo. The memory 40 of computer 36 may hold a data table 96 providing empirically determined load values 100 in three directions, torque, and a center of pressure (CoP) of the knee joint indexed according to output voltage (Vₒᵤₜ). Additional fields 98 may also hold knee joint characteristics, for example, aspect ratio, femoral anteroposterior and mediolateral width, and the tibial anteroposterior and mediolateral width or the like.

For an applied load, the physician may receive a center of pressure 106 and corresponding load values 100, in three directions, for the center of pressure 106 location for review by a physician on the display 43 and to balance the contact forces during TKA and UKA as can be determined by visual inspection.

Generally, the center of pressure 106 is the location of the average load value 100. Trained algorithms may be used to predict center of pressure 106 based on a functional relationship between the load value, location of contact forces or pressures, and the resulting output voltage (Vₒᵤₜ). For example, the memory 40 of computer 36 may hold a data table 96 providing empirically determined center of pressure 106 at a specific location of the knee joint indexed according to output voltage (Vₒᵤₜ) that can be references based on output voltage at different gauges. A center of pressure 106 location may be indicated numerically, for example, as coordinates, as a distance from a reference or control position in the medial-lateral and anterior posterior directions, or graphically on an image of an outline the knee and sensor 102 by a symbol 108 such as a dot or star at the center of pressure 106.

Trained algorithms may be used to predict load values 100, in three directions (i.e., medial-lateral (M-L) force, compression-distraction (C-D) force, anterior-posterior (A-P) force) and torque based on a functional relationship between the load values, location of loads, and the resulting output voltage (Vₒᵤₜ). For example, the memory 40 of computer 36 may hold a data table 96 providing empirically determined load values 100 at the center of pressure 106 indexed according to output voltage (Vₒᵤₜ).

The joint depiction may be labeled, for example, with a label indicating whether it is the lateral or medial compartment or a right or left side of the patient. Other desired or normal values including stress, tension, shear, and the like may alternatively or in addition be provided.

The outline of the knee and sensor 102 may allow for simultaneous display of the outline of the knee and sensor 102, e.g., top view of the knee joint, location of the sensor, and the applied loads in, e.g., three directions (i.e., medial-lateral (M-L) force, compression-distraction (C-D) force, anterior-posterior (A-P) force) at a center of pressure (CoP), and in this way, force on the different compartments of the knee joint may be easily viewed and distinguished. By reviewing the applied loads in real time, the physician can perform, e.g., sagittal plane balancing, coronal plane balancing, or balancing of other planes, and may make any adjustments or corrections to obtain desired forces throughout flexion to ensure that the soft tissues are properly tensioned across the full flexion range.

Additional applications of the load measuring device 10 may include use with a knee joint distractor or tensor whereby the joint surfaces are pulled apart and the bones held in place with pins in an external fixation frame allowing damaged cartilage to repair itself. In this respect the load measuring device 10 may assist with estimating ligament slack lengths, performing validation using additional loading scenarios, and evaluating sensitivity to the applied loads for applications including intraoperative computational modeling.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a controller” and “a processor” can be understood to include one or more microprocessors that can communicate in a stand-alone and/or a distributed environment(s) and can thus be configured to communicate via wired or wireless communications with other processors, where such one or more processors can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

The terms “load value”, “load measurement”, and “load measure” are intended to generally describe measurements of force and load including but not limited to stress or tension.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112 (f) unless the words “means for” or “step for” are explicitly used in the particular claim.

What we claim is:

1. A system for measurement of loading in a joint of a real or artificial limb extending along a longitudinal axis, the system comprising:
a load measuring device insertable into the joint and supporting:
a first force sensor detecting a load input on the joint along a first axis and producing a first output voltage indicating at least one of a normal and shear force on the joint;
a second force sensor detecting the load input on the joint along a second axis displaced from the first axis and producing a second output voltage indicating the at least one of a normal and shear force on the joint;
a third force sensor detecting the load input on the joint along a third axis displaced from the first and second axis and producing a third output voltage indicating the at least one of a normal and shear force on the joint;
a machine learning system having a processing circuit receiving the first, second, and third output voltages to determine and output an output value functionally related to a loading on the joint and based on a predetermined relationship between the first, second, and third output voltages and the load input determined using a machine learning training set.

2. The system of claim 1 wherein the output value provides a load measurement of at least one of a normal force, shear force, and a center of pressure of the joint.

3. The system of claim 2 further including an output device outputting a file for storage linking the output voltage with the output value based on the relationship between the output voltages and the loading on the joint at a load location.

4. The system of claim 1 further including an output device outputting a file for storage linking the output voltage with a center of pressure based on the relationship between the output voltages and the loading on the joint at a load location and further including a display simultaneously indicating a location of the center of pressure on the joint.

5. The system of claim 1 wherein the machine leaning training set comprises a load input data set at associated load locations and an output voltage data set.

6. The system of claim 1 wherein the load measuring device comprises a disk supporting the first force sensor, second force sensor, and third force sensor displaced apart on the disk.

7. The system of claim 6 wherein the disk comprises a ring formed of at least three beams, each supporting one of the first force sensor, second force sensor, and third force sensor thereon.

8. The system of claim 7 further comprising
a fourth force sensor detecting force on the joint along a fourth axis displaced from the first, second and third axes and producing a fourth voltage indicating the at least one of a normal and shear force on the joint;
wherein the machine learning system receives the fourth voltage of the fourth force sensor.

9. The system of claim 8 wherein the disk comprises a ring of at least four beams, each supporting a force sensor thereon.

10. The system of claim 9 further comprising
a fifth force sensor detecting force on the joint along a fifth axis displaced from the first, second, third and fourth axes and producing a fifth voltage indicating the at least one of a normal and shear force on the joint;
wherein the machine learning system receives the fifth voltage of the fifth force sensor;
a sixth force sensor detecting force on the joint along a sixth axis displaced from the first, second, third, fourth, and fifth axes and producing a sixth voltage indicating the at least one of a normal and shear force on the joint;
wherein the machine learning system receives the sixth voltage of the sixth force sensor.

11. The system of claim 10 wherein the disk comprises a ring of at least six beams, each supporting at least one force sensor thereon.

12. The system of claim 11 wherein the ring supports a top, bottom, left side and right side beam and first and second cross beams intersecting the top, bottom, left side and rights side beams.

13. The system of claim 6 wherein the first, second and third sensors detect a normal force on the joint.

14. The system of claim 13 wherein at least one of the first, second and third sensors detect a shear force in a first direction and a second shear force in a second direction on the joint.

15. The system of claim 14 wherein the first, second and third sensors detect a center of pressure of the joint.

16. The system of claim 6 wherein the first, second, and third force sensors each comprise of one or more strain gauges.

17. The system of claim 16 wherein the disk comprises a ring of at least three beams wherein each of the at least three beams supports at least two upper strain gauges and at least two lower strain gauges.

18. The system of claim 17 wherein the at least two upper strain gauges and the at least two lower strain gauges are wired through at least one Wheatstone bridge circuit.

19. The system of claim 18 wherein the at least one Wheatstone bridge circuit is a full, half or quarter Wheatstone bridge circuit.

20. A method of measuring joint forces for a joint replacement comprising the steps of:
(a) installing a load measuring device into a patient; and
(b) making measurement of a load input on the joint interface by:
(i) applying a load to the load measuring device;
(ii) detecting a first normal or shear force on the load measuring device at a first sensor along a first axis;
(iii) detecting a second normal or shear force on the load measuring device at a second sensor along a second axis displaced from the first axis;
(iv) detecting a third normal or shear force on the load measuring device at a third sensor along a third axis displaced from the first and second axes;
(v) applying a predetermined relationship between the first, second, and third output voltages at the first, second and third sensors, respectively, and the load input determined by machine learning; and
(vi) processing the first, second, and third output voltages at the first, second and third sensors, respectively, to output a value functionally related to the load input applied to the load measuring device.
